(19) United States
(12) Patent Application Publication
(10) Pub. No.: US 2011/0059482 A1
(43) Pub. Date: Mar. 10, 2011

(54) Sampling Kit

(76) Inventor: Hector Roldan, Tyreso (SE)

(21) Appl. No.: 12/921,189

(22) PCT Filed: Mar. 12, 2009
(86) PCT No.: PCT/SE09/00135

§ 371 Date: Oct. 19, 2010

Related U.S. Application Data
(60) Provisional application No. 61/036,029, filed on Mar. 12, 2008.

(30) Foreign Application Priority Data
Mar. 12, 2008 (SE) 0800569-6

(51) Int. Cl. C12Q 1/14; C12M 1/34; C12Q 1/04; C12Q 1/10

(52) U.S. Cl. 435/36; 435/287.1; 435/34; 435/38

(57) ABSTRACT

The present invention relates to a kit for improving and facilitating sampling and handling of biological samples from sampling to analysis. The invention also relates to a method for selection and, optionally, enrichment of microbes for efficient and accurate analysis of a biological sample as well as a collection vessel.

### FIELD OF THE INVENTION

The present invention relates to a kit for use in situations where a biological sample is taken, and in particular when the sample requires pre-treatment before analysis. The components and the use of the kit permit a significant shortening of the handling time from sampling to analysis. Further, the invention relates to a method for enrichment and selection of microbes as well as a collection vessel for sampling and selection of microbes.

### BACKGROUND OF THE INVENTION

Antibiotic resistance can cause serious disease and is an important public health problem. Over time, some bacteria have developed ways to circumvent the effects of antibiotics. Widespread use of antibiotics is thought to have driven evolutionarily adaptations that enable bacteria to survive these drugs. Other microbes such as viruses, fungi, and parasites have developed resistance as well. Antimicrobial resistance provides a survival benefit to microbes and makes it harder to eliminate infections from the body. Drug resistance is an especially difficult problem for hospitals treating critically ill patients who are less able to fight off infections without the help of antibiotics. Heavy use of antibiotics in these patients selects for changes in bacteria that bring about drug resistance. Unfortunately, this worsens the problem by producing bacteria with greater ability to survive even in the presence of the strongest antibiotics available. Ultimately, the increasing difficulty in fighting off microbes leads to an increased risk of acquiring infections in hospitals or other settings.

The most serious concern with antibiotic resistance is that some bacteria have become resistant to almost all of the easily available antibiotics and are able to cause serious disease. Important examples of antibiotic resistant bacteria are methicillin-resistant Staphylococcus aureus (MRSA), vancomycin-resistant Enterococcus (VRE), multi-drug-resistant Mycobacterium tuberculosis (MDR-TB), extended spectrum β lactamase (ESBL) producing bacteria, Bordetella pertussis, Neisseria gonorreae and genital Myco-/Ureaplasma.

Transmission of antibiotic resistant bacteria from patient to patient in hospital settings is common. Contact with contaminated hands of hospital staff, contact with contaminated surfaces such as door handles, over bed tables and call bells and contact with contaminated equipment are examples of routes that spread antibiotic resistant organisms. Thus, standard precautions for health care facilities for the care of all patients, regardless of their diagnosis or presumed infection status, are crucial. They include good personal hygiene, the use of barrier equipment such as gloves, gowns, masks and goggles, appropriate handling and disposal of clinical waste and aseptic techniques.

Another important measure to prevent spreading of multi resistant microbes is monitoring and screening of patients and personnel in clinical health care settings. Usually several samples are collected from each person, e.g. from the throat/mouth, nose and perineum. This leads to an extremely large number of samples that need to be handled and analysed by the laboratories. In order to minimise the number of samples, samples from one patient are sometimes pooled in a new collection tube. In case of a positive analysis, new samples, which are not pooled, need to be sampled taken from the person in order to find the infectious spot of the body.

When the samples reach the laboratory, the laboratory personnel needs to sort, register and identify all samples originating from one person in order to pool the samples. It is common that one or more samples are difficult to find or identify. Once identified, the samples are transferred and pooled into a new tube wherein the incubation, selection and enrichment of bacteria take place. Incubation is usually performed over night before the analysis is made. Depending on the microbe to be screened for, analysis is made by DNA analysis or by further culturing and selection procedures.

All together, sampling, transportation and handling of samples and collection tubes are very costly and time consuming. The risk of errors also increases when handling a large number of samples. Further, it generates large amounts of disposals and clinical wastes that need to be taken care of. Thus, methods and means facilitating sampling and handling of samples at clinical care settings would be of great economical value.

The present invention provides a kit and method for minimising the number of collection tubes and reducing the time from sampling to analysis. The present invention also relates to a collection vessel for sampling and selection of antibiotic resistant microbes.

### SUMMARY OF THE INVENTION

The present invention relates to a kit improving and facilitating biological sampling and handling before analysis. The kit comprises at least one collection vessel for receiving at least one sample, a growth medium and at least one enzyme. The vessel has a closure for closing an open end of the vessel and the growth medium contained in the vessel is a gel based medium separated from the at least one enzyme. The at least one enzyme is an enzyme capable of digesting the gel based medium to a liquid growth medium upon coming into contact therewith.

The present invention also relates to a method for selection of microbes in a sample collected from a subject characterised in that the selection process starts in the collection vessel. The method comprises the following steps:
collecting at least one biological sample in a collection vessel comprising a growth medium, wherein the growth medium is a gel based growth medium,
adding at least one enzyme to the collection vessel, said enzyme dissolving said gel based medium to a liquid medium, and
selecting for said microbes in the collection vessel before analysis.

Further, the present invention also relates to a collection vessel to be used for biological sampling, incubation and selection.

### DESCRIPTION OF THE DRAWINGS

The invention is further described in the description, examples and claims with reference to the attached figures in which:

FIG. 1 is a flow chart showing how MRSA samples are handled from sampling to analysis today.

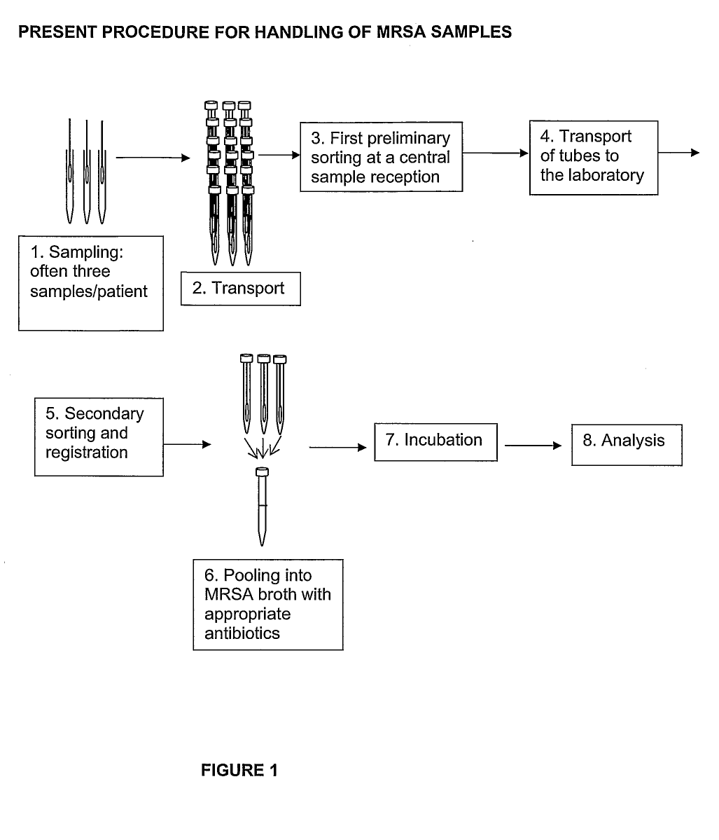

FIG. 2 is a flow chart showing how MRSA samples are handled from sampling to analysis in one embodiment of the present invention.

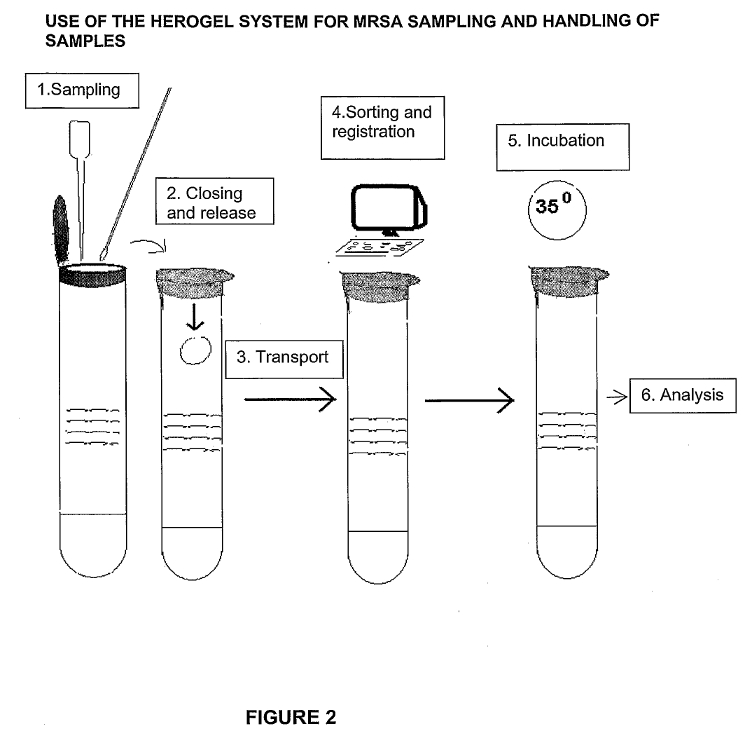

FIG. 3 illustrates a carrier (2) for a tablet (1) that can be placed in a collection device. The carrier (2) has flexible or elastic threads (3) upon which the tablet (1) is placed.

FIG. 4 illustrates an embodiment wherein a collection swab (8) is maintained in a collection vessel (4) after sampling. A and B illustrates before and after sampling. The collection swab (8) is placed in the collection vessel (4) through an open end (5). A tablet (1) is held in the collection device (4) by springing/elastic means (6, 7). The tablet (1) is released when the collection swab (8) is placed in the collection vessel (4). The collection swab (8) will be held in place and submerged in a medium (9) by the springing/elastic means (6,7) when the tablet (1) has been released.

FIG. 5 shows a specific embodiment wherein the collection swab (8) is maintained in the collection vessel (4) after sampling. A and B illustrates before and after sampling. The collection swab (8) is placed in the collection vessel (4) through the open end (5). The tablet (1) is held in the collection device (4) by a support (10). The tablet (1) is released when the collection swab (8) is placed in the collection vessel (4). The collection swab (8) will be held close to the vessel wall and submerged in the medium (9) by the springing/elastic means (6) when the tablet (1) has been released. An open space (11) makes it possible to collect a sample after incubation.

FIG. 6 schematically shows an embodiment of the collection vessel (4) wherein the closure (13) of the collection vessel (4) is adapted to release the enzyme upon closure of the vessel. A. Illustrates the collection vessel with an open end (5) before closure. The enzyme, in the form of a tablet (1), is included in the closure (13) by holding means (12). B. Illustrates the release of the tablet (1) upon closing the collection vessel (4). After release the tablet (1) will come into contact with the medium (9).

### DETAILED DESCRIPTION OF THE INVENTION

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise.

Also, the term “about” is used to indicate a deviation of +/−2% of the given value, preferably +/−5%, and most preferably +/−10% of the numeric values, where applicable.

In the context of the present invention the term “selection agent” relates to any compound or agent that can be used for treatment of a sample to obtain a desired result. Non-limiting examples of such desired results are selection, enrichment, microbial growth, inhibition, and transformation. Non-limiting examples of selection agents are antibiotics, inhibitors, growth inhibitors, colouring agents, growth stimulating compounds, transformation agents, genetic elements (transposons, plasmids, etc) that can be inserted into the genome of a desired microbe, and labelling agents.

In the context of the present invention the term “antibiotic” relates to compounds and compositions used to combat bacteria but also antimicrobial agents such as antifungal, antiviral and antiparasitic agents. An antifungal drug is medication used to treat fungal infections. Antiviral drugs are a class of medication used specifically for treating viral infections. Like antibiotics, specific antivirals are used for specific viruses. Antiparasitics are a class of medications which are indicated for the treatment of infection by parasites such as nematodes, cestodes, trematodes, infectious protozoa, and amoebas.

In the context of the present invention the term “digesting enzyme” relates to an enzyme capable of breaking down or dissolving a gelatinous substance in the gel based growth medium turning it in to a liquid medium.

In the context of the present invention the term “sampling” relates to the collection of any sample that is to be transported to a laboratory or the like for analysis.

In the context of the present invention the term “microbial sampling” relates to the collection of a sample comprising or suspected of comprising microbes. The sample can be collected from a subject, such as a human or an animal (e.g. a domestic animal or a wild animal) or from equipment and interior fittings of for example, but not limited to, hospitals and clinical health care settings, natural environments, veterinary settings, stables, animal cages, pigsties, poultry houses or the like.

In the context of the present invention the term “substantially simultaneously” means that a method step, takes place immediately before or after another step, or simultaneously therewith, e.g. the addition of the enzyme takes place after the sample has been deposited into the collection vessel, but before the closure or lid is closed.

As the use of antibiotics increases world wide, screening subjects for antibiotic resistant organisms will continue to be a concern at health care settings in order to minimise the spread of such organisms. The more frequent subjects are screened, the more efficient will the fight against these organisms be. The present invention provides means enabling time and cost efficient screening of subjects, such as patients and personnel, at health care settings.

The present invention relates to a kit for sampling of a biological sample containing or suspected of containing one or more microbes of interest, for example (but not limited to) bacteria, fungi and viruses. The kit comprises at least one collection vessel for receiving at least one sample, a growth medium, and at least one enzyme. The collection vessel has a closure for closing an open end of the vessel. The closure can be, but is not limited to, a screw cap, a snap lock, or a plug. The growth medium contained in the vessel is a gel based medium separated from the at least one enzyme and said at least one enzyme is an enzyme capable of digesting the gel based medium to a liquid growth medium upon coming into contact therewith.

Preferably the kit comprises at least one selection agent to be added to the collection vessel in order to obtain a desired result. One or more selection agents can be used simultaneously. If more than one selection agent is used, said selection agents can be a combination of different types of selection agents in order to achieve a specific result. The at least one selection agent may be separated from the growth medium or may be comprised in the growth medium. If the stability of a selection agent is short, the selection agent is preferably provided separated from the growth medium. In such case, the at least one selection agent is in dry form, semi-liquid or in liquid form. If unstable selection agents are kept separate from the growth medium and added to the collection vessel immediately before, upon or after sampling, the shelf life of the kit greatly increases. In one embodiment the growth medium is also the at least one selection agent.

Selection agents in dry form may be in or comprised in any suitable form for example, but not limited to, a powder, a tablet or the like, a bio disc eg a paper disc, a granulate, enclosed in a capsule or in microspheres, adhered to glass beads or beads of any suitable material. If the selection agent is in semi-liquid or in liquid form it can, for example, be enclosed in a capsule or the like that releases the selection agent in the collection vessel.

In one preferred embodiment the kit is for sampling of antibiotic resistant bacteria and the at least one selection agent is an antibiotic.

Any antibiotics suitable for selection and enrichment of antibiotic resistant microbes can be included in the kit, such as, but not limited to, beta-lactam antibiotics (such as cephalosporins), polymyxin antibiotics, cephamycin antibiotics, glycopeptide antibiotics, aminoglycosides, quinolons, macrolids, carbapenems and penicillins.

The growth medium is a broth adapted for the microbes that are to be detected. Broths for enrichment and/or selection of different microbes, such as bacteria and fungi, are well known for the skilled person and can easily be bought from chemical suppliers, for example, but not limited to, Oxoid Limited, Hampshire, UK; Becton & Dickinson, Stockholm, Sweden; and Merck AB, Stockholm, Sweden.

As mentioned above, the growth medium can contain one or more selection agent if stable in said medium. In Fang et al., J. Clin Microbiol, vol. 44, p. 592-594, 2006 (“Use of Cefoxitin-Based Selective Broth for Improved Detection of Methicillin-Resistant Staphylococcus aureus”) a selective broth for detection of MRSA is disclosed.

The growth medium of the inventive kit is preferably a gel based medium. A gel based medium facilitates the handling of the collection vessel and the sample upon sampling. It avoids spillage, splashing and leakage before and during sampling. The growth medium can, for example, be made gelatinous through the addition of, for example but not limited to, gelatine or agar. The concentration of gelatine is not more than about 50 mg/ml growth medium, preferably not more than about 20 mg/ml growth medium, more preferable not more than about 10 mg/ml growth medium, more preferable not more than about 5 mg/ml growth medium and even more preferable not more than about 2 mg/ml.

Preferably, the medium is in liquid form after sampling to increase the diffusion of selection agents and microbes. In one embodiment the gel based medium is turned into a liquid medium through the addition of an enzyme. The enzyme can be any enzyme capable of digesting a gel based medium into a liquid medium, such as, but not limited to, proteases (proteolytic enzymes, peptidases) and gelatinases. A more specific example of the enzyme is a gelatine digesting enzyme, such as, but not limited to bromelain (stem- or fruit-) or papain. The enzyme will be used in an amount of about 0.01-1000 ug/ml, preferably about 0.1-500 ug/mland more preferably about 5-200 ug/ml. As a non-limiting example, the enzyme activity of bromelanin is estimated to 0.33 Units/100 μg enzyme.

The enzyme can be in dry form, such as a powder, a tablet, a bio disc, a powder contained in microspheres, a powder adhered to glass beads or beads of any other suitable material.

The at least one enzyme and the at least one selection agent can be contained in the same tablet, microspheres or bio disc or in separate tablets, microspheres or separate bio discs. The at least one enzyme and the at least one selection agent can also be in the form of separate liquids or semi-liquids.

Optionally, the gel based medium comprises an antifoam agent. The antifoam agent is added to the medium in order to minimise the foaming which interferes with subsequent analysis procedures. An antifoam agent is a chemical that reduces the surface tension of foams that form on the surface of broths during incubation because of aeration or agitation. Non-limiting examples of antifoam agents are stearyldecanol, octal decanol, vegetable oils, silicones, sulphonates, and polypropylene glycol.

The inventive kit can be adapted for selection and optionally, enrichment, of different microbes. Non-limiting examples of microbes that can be selected for by the use of the present invention are methicillin resistant Staphylococcus aureus (MRSA), vancomycin-resistant Enterococcus (VRE), multi-drug-resistant Mycobacterium tuberculosis (MDR-TB), extended spectrum β lactamase (ESBL) producing bacteria, Bordetella pertussis, Neisseria gonorreae, genital Myco-/Ureaplasma, Mycoplasma pneumoniae, Streptococcus pneumoniae and beta hemolysing Streptococcus strains, different Eschericha coli strains such as enterohemorragic E. coli strains (EHEC), enterotoxigenic E. coli strains (ETEC), enteroinvasive E. coli strains (EIEC), enteropathogenic E. coli strains (EPEC) and enteroaggregative E. coli strains (EAggEC).

In one embodiment of the invention the kit is adapted for selection and, optionally, enrichment of MRSA. In this context, the term “adapted” means that the kit comprises selection agents and growth medium appropriate for selection and enrichment of MRSA. One example of selection agents that can be used is antibiotics. Examples of antibiotics that can be used for selection of MRSA are, but not limited to, cephalosporins such as Aztreonam®, Colistin®, Cefoxitin®, and Cefpodoxim®, methicillins, and penicillins such as isoxapenicillin. In one embodiment, at least one of Aztreonam® and Colistin® are included in the gel based medium and Cefoxitin® is added immediately before, upon, or after sampling. In another embodiment Cefoxitin® together with Aztreonam® and/or Colistin® is added immediately before, upon, or after sampling. Cefoxitin®, but also Aztreonam® and Colistin®, are relatively unstable antibiotics. Thus, adding at least one of the antibiotics to the growth medium immediately before, upon or directly after sampling increases the shelf life of the kit markedly. If unstable antibiotics are added to the growth medium immediately before, upon or directly after sampling the shelf life of the kit will increase from about 14 days to at least 6 months. In one embodiment antibiotics inhibiting growth of all microbes except MRSA are included in the kit. Non-limiting examples of such antibiotics are cephalosporins.

In another embodiment the inventive kit is adapted for selection of ESBL producing bacteria. In this context, the term “adapted” means that the kit comprises selection agents and growth medium appropriate for selection and optionally, enrichment of ESBL. One example of selection agents that can be used is antibiotics. Examples of antibiotics that can be used for selection of ESBL are, but not limited to cephalosporins such as, but not limited to, Cefotaxim®, Ceftazidim®, Ceftriaxon®, Cefepim®, Cefpodoxim®, Cefuroxim®, Cefurxim-axetil®, Ceftibuten® and Cefadroxil®. In case of an occurrence of an ESBL strain having an associated resistance mechanism towards other antibiotics, these antibiotics can be used for selection of ESBL. Non-limiting examples of such antibiotics are aminoglycosides, Trimetoprim®, Trimetoprim-sulfa®, quinolons, and Nitrofurantoin®.

In another embodiment the inventive kit is adapted for selection of VRE. In this context, the term “adapted” means that the kit comprises selection agents and growth medium appropriate for selection and enrichment of VRE. One example of selection agents that can be used is antibiotics. Examples of antibiotics that can be used for selection and enrichment of VRE are, but not limited to, Vancomycin® and Colistin®. In case of an occurrence of a strain resistant to beta-lactam antibiotics, antibiotics such as, but not limited to, ampicillin, Piperacillin-Tazobactam, and carbapenems, can be used to select for that specific strain. If it is a VanA VRE strain, also Teicoplanin® can be used.

In another embodiment the inventive kit is adapted for selection of Bordetella pertussis. In this context, the term “adapted” means that the kit comprises selection agents and growth medium appropriate for selection and, optionally, enrichment of Bordetella pertussis. One example of selection agents that can be used is antibiotics. Examples of antibiotics that can be used for selection and enrichment of Bordetella pertussis are, but not limited to, beta-lactam antibiotics.

In another embodiment the inventive kit is adapted for selection of Neisseria gonorreae. In this context, the term “adapted” means that the kit comprises selection agents and growth medium appropriate for selection and, optionally, enrichment of Neisseria gonorreae. One example of selection agents that can be used is antibiotics. Examples of antibiotics that can be used for selection and enrichment of Neisseria gonorreae are, but not limited to, Vancomycin®, Colistin®, Nystatin®, Trmetoprim® and Polymyxin® and the like.

In another embodiment the inventive kit is adapted for selection of Myco-/Ureaplasma. In this context, the term “adapted” means that the kit comprises selection agents and growth medium appropriate for selection and, optionally, enrichment of Myco-/Ureaplasma. One example of selection agents that can be used is antibiotics. Examples of antibiotics that can be used for selection and enrichment of Myco-/Ureaplasma are, but not limited to, different types of penicillins. Antibiotics directed to the cell wall are ineffective to these microbes since they do not have a cell wall.

In another embodiment the inventive kit is adapted for selection of Mycoplasma pneumoniae. In this context, the term “adapted” means that the kit comprises selection agents and growth medium appropriate for selection and, optionally, enrichment of Mycoplasma pneumoniae. One example of selection agents that can be used is antibiotics. Examples of antibiotics that can be used for selection and enrichment of Mycoplasma pneumoniae are, but not limited to, different types of penicillins. Antibiotics directed to the cell wall are ineffective to these microbes since they do not have a cell wall.

One embodiment of the invention is a device or collection vessel in which a sample is deposited and incubated before the desired analysis is performed. Another embodiment is a kit, containing in addition to the vessel also parts necessary for the sampling and deposition of the sample in the vessel.

In its most basic form, the collection vessel is a tube which is closed at one end and open at the opposite end, and includes means for closing the open end. Said tube can be a conventional test tube, but preferably is a tube having an internal structure which aids in the deposition and culturing (selection) of the sample. When transferring a sample into a collection vessel containing a growth medium and/or a selection medium, it is important that a substantial amount of the sample is indeed transferred onto/into the medium and that only an insubstantial amount remains on the swab or the like used for sampling.

To avoid the removal of any sample from the collection vessel upon the removal of the collection swab, the collection swab can be left in the collection vessel. If the swab or the like used for sampling is left in the collection vessel and the shaft of the swab is longer than the collection vessel, the shaft is simply broken off and adjusted to an appropriate length. If the swab or the like used for sampling is left in the collection tube after sampling it is important that the part of the swab comprising the sample is properly submerged in the medium. In one embodiment the collection vessel comprises means for maintaining the swab in the medium. Such means can for example be protrusions from the vessel wall between which the shaft of the swab can be fixed and held at a specific position. Other means keeping the shaft of the swab in a position where the swab is submerged into the medium, are for example, but not limited to, elastic or springing means or means other for pressing the swab towards the vessel wall to keep it in a fixed position. When the shaft and the swab are fixed or held in a specific position towards the wall of the collection vessel, removing a certain volume of the incubated sample is also facilitated. Thus, the means for fixing or holding the collection swab close to the vessel wall and/or submerged in the growth medium should preferably have a shape and form that do not interfere or inhibit the removal of a sample for analysis. For example, if the incubated sample is to be analysed by an automated system the shaft of the swab is preferably fixed or held close to the wall of the collection vessel in order to facilitate removal of a certain volume of the sample with a tip of the automated system. Non-limiting examples of such shape and form of the means holding the collection swab in a specific position are the form of a ring, bow, arch, curve or the like. The means for maintaining the swab in the medium and/or holding it towards the vessel wall is an internal structure that can be an integral part of the vessel, a detail added to the vessel after its manufacture or a separate mould also added to the vessel after its manufacture.

In a procedure where the swab is not left in the medium, it frequently happens that medium and part of the sample adheres to the swab and, consequently some medium and sample are removed from the collection vessel together with the swab. This problem is addressed in an embodiment of the invention where the collection vessel has an internal structure, making it possible to scrape off sample and medium possibly adhering to the collection swab. This internal structure can be an area on the inside of the vessel, said area having a roughened surface, protrusions, rills or the like. This internal structure can be an integral part of the vessel, or a detail added to the vessel after its manufacture.

The collection vessel of the invention can also be designed with a non-symmetrical distal end or other means which aids in the positioning of the vessel in for example a tube rack or block having corresponding depressions for receiving the vessel.

The at least one enzyme and/or the at least one selection agent included in the kit is/are added to the collection vessel substantially simultaneously with the sample. The at least one enzyme and/or the at least one selection agent can be added to the collection vessel manually immediately before or after the sampling. However, the collection vessel can also be adapted to release said at least one enzyme and/or said at least one selection agent immediately before or after the sampling.

The collection vessel can be adapted to contain at least one compound in a carrier or support and said carrier or support releases said at least one compound when the collection swab is placed in the collection vessel. Preferably the placement of the collection swab in the collection device leads to the release of said compound from the carrier or support. This can be achieved by including the at least one compound in the form of a tablet or a capsule or the like that can be deposited on or contained in the carrier or support. In one embodiment the carrier or support can also be the elastic/springing means. Preferably, the at least one compound is the at least one enzyme and/or the at least one selection agent mentioned above in the form of a tablet, capsule or the like. The carrier or support adapted to contain at least one compound is an internal structure that can be an integral part of the vessel, or a detail added to the vessel after its manufacture or a separate mould also added to the vessel after its manufacture.

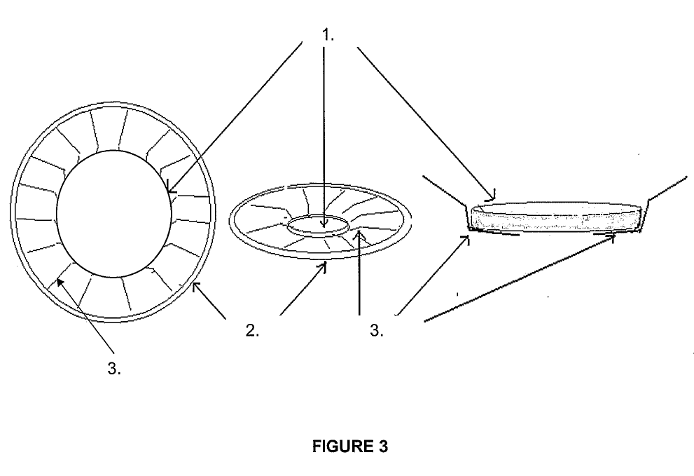

FIG. 3 illustrates a specific embodiment of a carrier that can be a separate part added to the collection vessel after its manufacture or an integral part of the vessel. The carrier has flexible or elastic threads or fibres upon which the tablet is placed. The tablet is released upon deposition of the sample in the collection vessel with the collection swab, i.e. the collection swab pushes the tablet into the medium as it is pushed through the carrier. If the collection swab is removed from the collection vessel the flexible or elastic threads or fibres of the carrier will also function as an internal structure scraping of sample and medium adhering to the collection swab. The sample and medium will subsequently melt as the collection vessel is incubated at a temperature above room temperature and thereby drip into the medium in the bottom of the collection vessel. The carrier should preferably be positioned at a certain distance (at least about 1 cm) above the medium in order to avoid that the tablet gets wet or moistened if the medium melts prior to sampling for example due to a high temperature in the room wherein the collection vessel is maintained prior to its use.

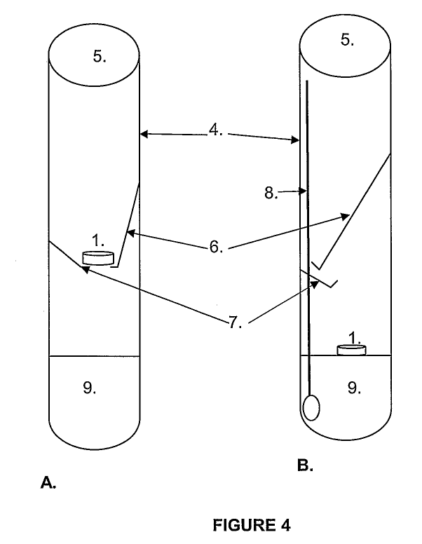

FIG. 4 illustrates an embodiment wherein the collection swab is maintained in the collection vessel after sampling. The collection swab is placed in the collection vessel through the open end. The tablet is held in the collection device by springing/elastic means. The tablet is released when the collection swab is placed in the collection vessel, i.e. the collection swab pushes the springing/elastic means apart by pushing the tablet downwards towards the closed end of the collection vessel. The tablet is thereby released into the medium. The collection swab will be held in place towards one side of the collection vessel and submerged in the medium by the springing/elastic means when the tablet has been released.

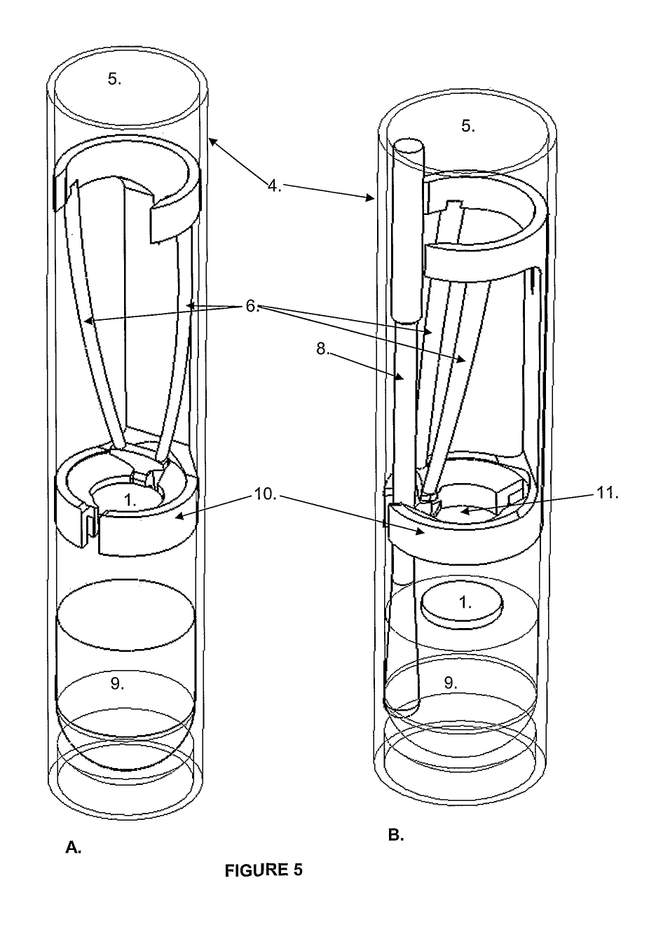

FIG. 5 shows a specific embodiment wherein the collection swab is maintained in the collection vessel after sampling. The collection swab is placed in the collection vessel through the open end. The tablet is held in the collection device by a support. The tablet is released when the collection swab is placed in the collection vessel, i.e. the collection swab pushes the tablet downwards and into the medium as it is pushed through the support. The collection swab will be held in a specific position and submerged in the medium by the springing/elastic means when the tablet has been released. The springing/elastic means pushes the collection swab towards one side of the collection well. An open space makes it possible to collect a sample after incubation. Such an open space particularly facilitates removal of an incubated sample by an automated system.

The closure of the collection vessel or device can also be adapted to contain at least one compound, preferably in such fashion that the at least one compound is released upon closing the tube. This can be achieved by including the at least one compound in the form of a powder, a tablet, capsule or the like in the cap, lock or plug. In one specific embodiment the closure of the vessel is adapted to contain the at least one enzyme and/or the at least one selection agent, preferably in such fashion that the at least one enzyme and/or the at least one selection agent is/are released upon closing the tube. This can be achieved by including the at least one enzyme and/or the at least one selection agent in the form of a powder, a tablet, capsule or the like in the closure, cap, lock or plug. Preferably the at least one enzyme and/or the at least one selection agent is/are contained in the cap, lock, or plug and not released until the cap, lock or plug effectively closes the vessel, or optionally upon activation of a release mechanism. Such release mechanism can be a breakable seal, a plunger, an openable lid etc. The selection agent and/or enzyme, when in dry form, can be enclosed in a compartment which opens upon application of pressure. The selection agent and/or enzyme, when in tablet form, can be in a press-fit engagement, held in place in the lock by a rill, a velt or other protrusions or holding means, and dislocated by the pressure when the cap, lock or plug is fastened or inserted to close the vessel.

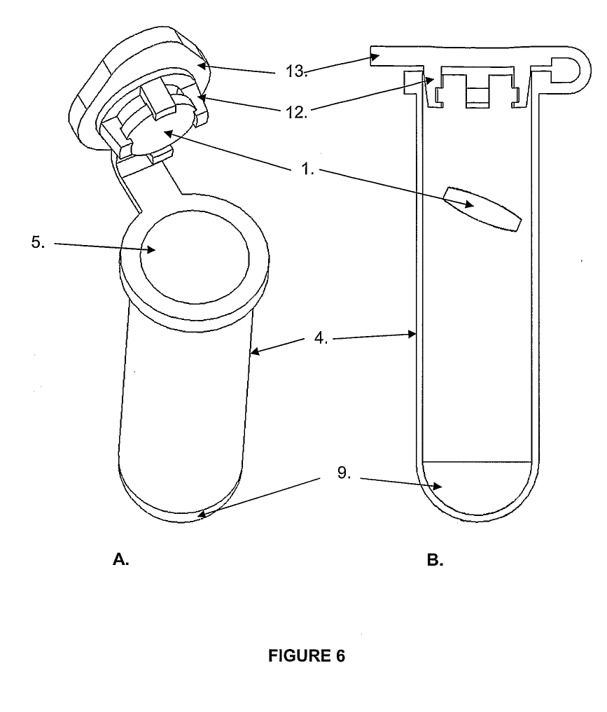

FIG. 6 shows an example of a specific embodiment wherein a release mechanism is incorporated in the closure. The closure of the collection vessel is adapted to release the enzyme and/or selection agent, preferably in the form of a tablet upon closure of the vessel. The tablet is contained in the closure by holding means and released from the closure into the medium upon closing the collection vessel. The holding means are pushed outwards upon closure of the collection vessel. This can, for example, be achieved by flexible means in the closure or the closure being flexible itself which forces the holding means outwards when the collection vessel is closed and the flexible means or the flexible closure is pushed or pressed downwards. When the holding means are pushed outwards the tablet is released from the holding means.

Another embodiment of the invention is a kit comprising at least one collection vessel, closure, growth medium, and enzyme, including the selection agent either incorporated in the growth medium or with the possibility to be added to the same. According to yet another embodiment the kit also comprises a collection swab. The collection swab can be any device suitable for collecting a biological sample, such as swabs that are routinely used at clinical health care settings today. In one embodiment the collection swab is a shaft with a tip of suitable material, such as a cotton bud, a sponge, a brush, a serrated tip etc.

The kit can also comprise a pipette for taking a defined volume of a liquid sample, such as but not limited to a urinary sample. Preferably the pipette has a volume which, together with the amount of growth medium, ensures a repeatable and standardized sample volume. It is important that the volume of liquid sample is standardized so that the concentration of selection agent(s) in the sample will not be too high if the sample volume is smaller than expected or too low if the sample volume is higher than expected. In one non-limiting example the liquid sample volume added to 1 ml growth medium is 50 ul. In addition to pipettes, collection swabs with tips adapted for liquid sampling can also be used.

Presently, samples collected for different analyses are sampled in identical or similar collection vessels. This complicates the sorting and identification of samples and makes this procedure very time consuming and prone to errors. Therefore the closure of the vessel of the present invention can be designed to facilitate the identification of the microbes that are to be selected for by the use of the collection vessel. The closure can for example have a specific colour for selection of a specific microbe. As an alternative or as a complement, signs or text can be printed on the closure of the vessel, such as the name of the microbe or group of microbes that are to be selected for by the use of the kit.

The present invention also relates to a method for selection and, optionally, enrichment of microbes in a sample collected from a subject, such as an animal or a human. The method enables the selection process to start already in the collection vessel. The method comprises the following steps:
collecting at least one biological sample comprising or suspected of comprising the microbes that are to be selected for in a collection vessel comprising a growth medium, wherein the growth medium is a gel based growth medium,
adding at least one enzyme to the collection vessel, said enzyme dissolving said gel based medium to a liquid medium, and
selecting for said microbes in the collection vessel before analysis.

The inventive method is for sampling of a sample containing or suspected of containing a specific microbe or microbes and selection and, optionally, enrichment of said microbes in the collection vessel. The growth medium contained in the vessel is a gel based medium separated from the at least one enzyme and said at least one enzyme is an enzyme capable of digesting the gel based medium to a liquid growth medium upon coming into contact therewith.

The enzyme ensures that the gelatine becomes and remains in liquid form during incubation and analysis. In order to decrease the time from sampling to analysis, the collected sample can be subjected to heat. The heat will for example make the enzyme more active in a shorter period of time compared to if heat is not applied. In addition, also the selection of bacteria will start earlier if heat is applied since the selection agent will be spread faster in the medium when the medium is in liquid form. As a non-limiting example, if the enzyme is added in the form of a tablet and the collection vessel is incubated at a temperature of about 35-40° C. for about two hours the enzyme ensures that the gelatine remains in liquid form throughout incubation and analysis. Suitable temperatures for enzyme activation and microbe selection are well known to the person skilled in the art.

Shaking of the collection vessel after sampling and after addition of the enzyme will also reduce the time to activate the enzyme as well as the selection of microbe(s). The shaking should be gentle and correspond to a frequency and intensity of shaking usually applied in the laboratory when culturing microbes. Such frequency and intensity of shaking is well known to the skilled person.

Preferably at least one selection agent is added to the collection vessel, said at least one selection agent is dissolved in the growth medium and is added in order to obtain a specific result. One example of such a result is selection of a specific microbe.

The selection agent may be separated from the growth medium or may be contained in the growth medium. If the stability of the at least one selection agent is short, the selection agent is preferably added to the growth medium immediately before, upon or after sampling. In such case, the selection agent is in dry form or in liquid or semi-liquid form. In one embodiment the growth medium is also the at least one selection agent.

Selection agents in dry form may be in or comprised in any suitable form for example, but not limited to, a powder, a tablet or the like, a bio disc eg a paper disc, a granulate, enclosed in a capsule or microspheres, adhered to glass beads or beads of any suitable material. If the selection agent is in semi-liquid or in liquid form it can, for example, be enclosed in a capsule or the like that releases the selection agent in the collection vessel.

In one embodiment the method is for selection of antibiotic resistant bacteria. For selection of antibiotic resistant bacteria one or more antibiotics are used.

Any antibiotics suitable for selection and enrichment of antibiotic resistant microbes can be used in the method, such as, but not limited to, beta-lactam antibiotics (such as cephalosporins), polymyxin antibiotics, cephamycin antibiotics, glycopeptide antibiotics, aminoglycosides, quinolons, macrolids, carbapenems and penicillins.

The growth medium is a broth adapted for the microbes that are to be detected. Broths for selection and enrichment of different microbes, such as bacteria, are well known for the skilled person and can easily be bought from chemical suppliers. Examples of chemical suppliers are, but not limited to, Oxoid Limited, Hampshire, UK; Becton & Dickinson, Stockholm, Sweden; and Merck AB, Stockholm, Sweden.

As mentioned above, the growth medium can contain one or more selection agents if stable in said medium. In Fang et al., J. Clin Microbiol, vol. 44, p. 592-594, 2006 (“Use of Cefoxitin-Based Selective Broth for Improved Detection of Methicillin-Resistant Staphylococcus aureus”) a selective broth for detection of MRSA is disclosed.

The growth medium of the inventive method is preferably a gel based medium. A gel based medium facilitates the handling of the collection vessel and the sample upon sampling. It avoids spillage, splashing and leakage before and during sampling. In one embodiment the growth medium is made gelatinous through the addition of for example gelatine or agar. The concentration of gelatine is not more than about 50 mg/ml growth medium, preferably not more than about 20 mg/ml growth medium, more preferable not more than about 10 mg/ml growth medium, more preferable not more than about 5 mg/ml growth medium and even more preferable not more than about 2 mg/ml.

Preferably, the medium is in liquid form after sampling to increase the diffusion of selection agents and the microbes. The gel based medium can be turned into a liquid medium through the addition of an enzyme. The enzyme can be any enzyme capable of digesting a gel based medium into a liquid medium, such as proteases (proteolytic enzymes, peptidases) and gelatinases. In one embodiment the enzyme is a gelatine digesting enzyme, such as, but not limited to bromelain (stem- or fruit-) or papain. The enzyme will be used in an amount of about 0.01-1000 ug/ml, preferably about 0.1-500 ug/mland more preferably about 5-200 ug/ml.

Examples of dry forms of the enzyme are, but not limited to, a powder, a tablet, a bio disc, a powder contained in microspheres, powder adhered to glass beads or beads of any other suitable material. The enzyme can also be in a liquid or semi-liquid form.

The at least one enzyme and the at least one selection agent can be contained in the same tablet, bio disc, microspheres or capsules or in separate tablets, separate bio discs, microspheres or capsules. The at least one enzyme and the at least one selection agent can also be in the form of separate liquids. Thus, the at least one enzyme and the at least one selection agent can be added to the growth medium together, simultaneously or separate.

An antifoam agent is added to the medium in order to minimise the foaming which interferes with subsequent analysis procedures. An antifoam agent is a chemical that reduces the surface tension of foams that form on the surface of broths during incubation because of aeration or agitation. Non-limiting examples of antifoam agents are stearyldecanol, octal decanol, vegetable oils, silicones, sulphonates, and polypropylene glycol.

The inventive method can be adapted for selection and optionally, enrichment, of different microbes. Non-limiting examples of microbes that can be selected for and optionally enriched by the use of the present invention are methicillin resistant Staphylococcus aureus (MRSA), vancomycin-resistant Enterococcus (VRE), multi-drug-resistant Mycobacterium tuberculosis (MDR-TB), extended spectrum β lactamase (ESBL) producing bacteria, Bordetella pertussis, Neisseria gonorreae, genital Myco-/Ureaplasma, Mycoplasma pneumoniae, Streptococcus pneumoniae and beta hemolysing Streptococcus strains, different Eschericha coli strains such as enterohemorragic E. coli strains (EHEC), enterotoxigenic E. coli strains (ETEC), enteroinvasive E. coli strains (EIEC), enteropathogenic E. coli strains (EPEC) and enteroaggregative E. coli strains (EAggEC).

In one embodiment the method is for selection and, optionally, enrichment of MRSA. In this context, the term “adapted” means that the kit comprises selection agents and growth medium appropriate for selection and enrichment of MRSA. One example of selection agents that can be used is antibiotics. Examples of antibiotics that can be used for selection of MRSA are, but not limited to, cephalosporins such as Aztreonam®, Colistin®, Cefoxitin®, and Cefpodoxim®, methicillins, and penicillins such as isoxapenicillin. In one embodiment, at least one of Aztreonam® and Colistin® are included in the gel based medium and Cefoxitin® is added immediately before, upon, or after sampling. In another embodiment Cefoxitin® together with Aztreonam® and/or Colistin® is added immediately before, upon, or after sampling. Cefoxitin®, but also Aztreonam® and Colistin®, are a relatively unstable antibiotics. In one embodiment antibiotics inhibiting growth of all microbes except MRSA are included in the kit. Non-limiting examples of such antibiotics are cephalosporins.

In another embodiment the inventive method is for selection of ESBL producing bacteria. selection agents and growth medium appropriate for selection and optionally, enrichment of ESBL. One example of selection agents that can be used is antibiotics. Examples of antibiotics that can be used for selection of ESBL are, but not limited to cephalosporins such as, but not limited to, Cefotaxim®, Ceftazidim®, Ceftriaxon®, Cefepim®, Cefpodoxim®, Cefuroxim®, Cefurxim-axetil®, Ceftibuten® and Cefadroxil®. In case of an occurrence of an ESBL strain having an associated resistance mechanism towards other antibiotics, these antibiotics can be used for selection of ESBL. Non-limiting examples of such antibiotics are aminoglycosides, Trimetoprim®, Trimetoprim-sulfa®, quinolons, and Nitrofurantoin®.

In another embodiment the inventive method is for selection of VRE. In this context, the term “adapted” means that the kit comprises selection agents and growth medium appropriate for selection and enrichment of VRE. One example of selection agents that can be used is antibiotics. Examples of antibiotics that can be used for selection and enrichment of VRE are, but not limited to, Vancomycin® and Colistin®. In case of an occurrence of a strain resistant to beta-lactam antibiotics, antibiotics such as, but not limited to, ampicillin, Piperacillin-Tazobactam, and carbapenems, can be used to select for that specific strain. If it is a VanA VRE strain, also Teicoplanin® can be used.

In another embodiment the inventive method is for selection of Bordetella pertussis. In this context, the term “adapted” means that the kit comprises selection agents and growth medium appropriate for selection and, optionally, enrichment of Bordetella pertussis. One example of selection agents that can be used is antibiotics. Examples of antibiotics that can be used for selection and enrichment of Bordetella pertussis are, but not limited to, beta-lactam antibiotics.

In another embodiment the inventive method is for selection of Neisseria gonorreae. In this context, the term “adapted” means that the kit comprises selection agents and growth medium appropriate for selection and, optionally, enrichment of Neisseria gonorreae. One example of selection agents that can be used is antibiotics. Examples of antibiotics that can be used for selection and enrichment of Neisseria gonorreae are, but not limited to, Vancomycin®, Colistin®, Nystatin®, Trmetoprim® and Polymyxin® and the like.

In another embodiment the inventive method is for selection of Myco-/Ureaplasma. In this context, the term “adapted” means that the kit comprises selection agents and growth medium appropriate for selection and, optionally, enrichment of Myco-/Ureaplasma. One example of selection agents that can be used is antibiotics. Examples of antibiotics that can be used for selection and enrichment of Myco-/Ureaplasma are, but not limited to, different types of penicillins. Antibiotics directed to the cell wall are ineffective to these microbes since they do not have a cell wall.

In another embodiment the inventive method is for selection of Mycoplasma pneumoniae. In this context, the term “adapted” means that the kit comprises selection agents and growth medium appropriate for selection and, optionally, enrichment of Mycoplasma pneumoniae. One example of selection agents that can be used is antibiotics. Examples of antibiotics that can be used for selection and enrichment of Mycoplasma pneumoniae are, but not limited to, different types of penicillins. Antibiotics directed to the cell wall are ineffective to these microbes since they do not have a cell wall.

The collection vessel to be used in the inventive method can be any collection vessel suitable for sampling and selection of microbes. In one embodiment a collection vessel as described above is used in the inventive method.

In yet another embodiment of the inventive method a kit as described above is used for sampling, selection and optionally enrichment of microbes, for example, but not limited to, antibiotic resistant bacteria.

As mentioned in the “background”-section, several samples are collected from each person that is to be screened for MRSA, from different sites of the body. These samples are subsequently pooled to minimise the number of samples that need to be analysed. If a samples turns out to be positive, new samples are collected from the person in question but this time the samples are not pooled. By the use of the present invention several alternatives are possible. One alternative is that the samples are pooled directly upon sampling. This means that one step in the handling of samples after sorting and registration disappears completely, which of course means that valuable time can be saved. Another alternative is that the samples are not pooled at any time during the procedure. Since the use of the present inventive kit and method leads to the saving of time it is possible to analyse all samples originating from one person. If one sample turns out to be positive, the infectious spot of the body can be identified without the need to take new samples from the infected person.

The invention will now be further described in the following non-limiting examples.

### EXAMPLES

### Example 1

### Time Study

60224 MRSA samples were handled by the laboratory at Karolinska University, Solna, Sweden in year 2006. To handle (sorting, labelling and registration) these samples, 1404 hours were spent.

A time study for sorting, labelling and registration was performed based on the use of the present invention. The samples to be sorted, labelled and registered had been sampled in a collection vessel according to the invention.

100 samples were selected. Samples with both electronic referrals and paper referrals were included in the study. The samples were sorted, labelled and registered.

Results: 100 samples were sorted, labelled and registered in 45 minutes.

Handling as many samples as was handled by the laboratory at Karolinska University 2006 would take 450 hours by the use of the collection vessel according to the present invention, meaning a shortening of the handling time by 951 hours, corresponding to a 68% decrease!

In addition to the time that is saved, the amount of samples that needs to be carried and moved by the laboratory personnel also decreases markedly. Each rack with collection vessels weighs about 5 kg. Further, disposals and wastes from the laboratory decrease from about 1230 kg to about 500 kg per year.

### Example 2

### Study of the Concentration of Cefoxitin in a Tablet Formulation

Background: Herogel is a selective growth medium comprising aztreonam and colistin to inhibit the growth of Enterobacteriaceae. Cefoxitin is added to the medium in order to inhibit the growth of MSSA (Methicillin Sensitive Staphylococcus Aureus). Cefoxitin has a short shelf life if it is added to the medium upon manufacturing of the medium and together with other antibiotics. Tests performed at Karolinska University Hospital Laboratory have shown that the activity of cefoxitin in liquid form is reduced after 14 days at about 8° C. (i.e. in a refrigerator) and that aztreonam and colistin if they are added to the growth medium are active for at least 4 months if stored at a temperature of about 8° C. (i.e. in a refrigerator). In order to prolong the shelf life of the Herogel, cefoxitin is incorporated in the tablet in Batch 1. The hypothesis is that the shelf life of the herogel will be prolonged if the tablet is added immediately after the sampling. The shelf life of the tablet is estimated to about 12 months in room temperature. The other components of the Herogel medium have a documented shelf life of at least 6 months upon storage in a refrigerator.

Aim: The aim of the study was to determine the concentration of cefoxitin in the tablet by studying the growth of MSSA and MRSA in the medium.

### Materials and Reagents:

MSSA—laboratory culture
MRSA—laboratory culture
Herogel medium (2 ml/tube)—gelatin (2%), antifoam (0.006%), CM0067 (2.5%), aztreonam (0.0008%) colistin (0.0008%) and the tablet comprising cefoxitin (0.008 mg/tablet) and bromelain (0.1 mg/tablet)
ISO-MRSA broth—(2.34%, NaCl 2%)
PBS (1 ml/tube)—(NaCl 0.8%, KCl 0.02%, KH₂PO₄ 0.012%, Na₂HPO₄ 0.091%)
CM0067 meat broth
Blood agar plates

### Pipettes 10-40 μl and 40-200 μl

Sterile glass beads

### Thermostat 35° C.

### Method:

Day 1: Colonies of bacteria were suspended in PBS to about 0.5 McFarland. The bacterial suspensions were further diluted in a 100 μl dilution series, 1⁻¹-1⁻³. The dilutions 1⁻² and 1⁻³ were cultured as controls with 20 μl on blood agar plates. 4 sets of tubes with Herogel, iso-MRSA and CM0067. The tubes were marked 1-4. 1 tablet of batch 1 was added to two of the tubes containing Herogel. Tube no 1 was incubated in 35° C. for 1 hour and tube no 2 was incubated in room temperature. 20 μl of the bacterial dilutions 1⁻² and 1⁻³ was added simultaneously to all tubes. All tubes were incubated in 35° C. over night.

Day 2: Samples from all tubes were cultured on blood agar plates. The results are shown in Table 1-3.

TABLE 1
Culturing of the control dilutions

| Dilution MSSA | Cfu | Dilution MRSA | Cfu |
|---|---|---|---|
| 1⁻² | 108 | 1⁻² | 110 |
| 1⁻³ | 9 | 1⁻³ | 12 |

TABLE 2
Culturing (20 μl from dilution 1⁻²) of all tubes incubated over night

| Tube | Cfu MSSA | Cfu MRSA |
|---|---|---|
| 1 (Herogel + tablet) | 0 | continuous layer |
| 2 (ISO.MRSA broth) | 0 | continuous layer |
| 3 (CM0067 without ab) | continuous layer | continuous layer |
| 4 (Herogel + tablet) | 0 | continuous layer |

TABLE 3
Culturing (20 μl from dilution 1⁻³) of all tubes incubated over night

| Rör | Cfu MSSA | Cfu MRSA |
|---|---|---|
| 1 (Herogel + tablet) | 0 | continuous layer |
| 2 (ISO.MRSA broth) | 0 | continuous layer |
| 3 (CM0067 without ab) | continuous layer | continuous layer |
| 4 (Herogel + tablet | 0 | continuous layer |

Conclusion: The tablet of Batch 1 comprises 8 μg cefoxitin. The current cefoxitin level in the Herogel medium should therefore be 4 μg/ml since there are 2 ml in each tube. The minimal inhibitory concentration (MIC) for MSSA according to the referens group for questions related to antibiotic (RAF) is 4 mg/l.

Consequently, this study shows that the tablet releases a sufficient amount of cefoxitin to inhibit growth of the MSSA-strain that was used in this study.

### Example 3

### Decomposition of a Tablet in to Different Growth Medium, ISO-MRSA and Herogel-Medium

Aim: To study the dissolution of a tablet in two media and possible formation of precipitate or the occurrence of undissolved tablet material.

### Materials and Reagents:

4 tablet formulations named Batch I, Batch II, Batch III, Batch IV
4 tubes containing ISO-MRSA broth (liquid), 2 ml/tube
4 tubes containing Herogel-medium (solid medium), 2 ml/tube
Gelatine digesting enzyme (0.1 mg/tablet)

### Pipettes

Agitating apparatus

### Thermostat 35° C.

### Tablet Composition:

Batch I: Lactose, povidone and magnesium stearate. The tablet is hard.
Batch II: Lactose, povidone and magnesium stearate. The tablet is soft.
Batch III: Lactose, povidone, maize starch and magnesium stearate. The tablet is hard.
Batch IV: Lactose, povidone, maize starch and magnesium stearate. The tablet is soft.

Method: Enzyme, 50 μl, was added to the tubes containing Herogel. One tablet of each batch was added to each type of tube (i.e. tubes comprising ISO-MRSA broth and tubes comprising Herogel medium). All tubes were placed in an agitating apparatus in a thermostat. The decomposition was monitored 4 times during one hour (each 15 minutes). After one hour the tubes were further incubated in the thermostat over night in the agitating apparatus. After 16 hours, the final monitoring was made. The dissolution of the tablet was estimated as percentage of the reduction of the size of the tablet, e.g. 50% dissolution means that half of the tablet was dissolved. The formulation of precipitate was estimated by comparing the cloudiness in the medium with the McFarland scale. The results are shown in Table 4 and 5.

TABLE 4
ISO-MRSA Broth

| | Time | | | | |
|---|---|---|---|---|---|
| | | | | | Over night |
| Batch | 15′ | 30′ | 45′ | 60′ | (16 h) |
| BATCH I | 10% | 40% | 100% | 100% | 100% |
| | >2 McF | ~2 McF | ~2 McF | 1-2 McF | ~1 McF |
| BATCH II | 70% | 100% | 100% | 100% | 100% |
| | >2 McF | >2 McF | ~2 McF | ~2 McF | ~2 McF |
| BATCH III | 70% | 100% | 100% | 100% | 100% |
| | >2 McF | >2 McF | >2 McF | >2 McF | >2 McF |
| BATCH IV | 50% | 70% | 100% | 100% | 100% |
| | >2 McF | >2 McF | >2 McF | >2 McF | >2 McF |

TABLE 5
Herogel medium

| | Time | | | | |
|---|---|---|---|---|---|
| | | | | | Over night |
| Batch | 15′ | 30′ | 45′ | 60′ | (16 h) |
| BATCH I | 10% | 40% | 100% | 100% | 100% |
| | >2 McF | ~2 McF | ~2 McF | 1-2 McF | ~1 McF |
| BATCH II | 70% | 100% | 100% | 100% | 100% |
| | >2 McF | >2 McF | ~2 McF | ~2 McF | ~2 McF |
| BATCH III | 70% | 100% | 100% | 100% | 100% |
| | >2 McF | >2 McF | >2 McF | >2 McF | >2 McF |
| BATCH IV | 50% | 70% | 100% | 100% | 100% |
| | >2 McF | >2 McF | >2 McF | >2 McF | >2 McF |

The tablet formulation of Batch I dissolves slow but results in less cloudiness.

The tablet formulation of Batch II dissolves quickly but results in a rather high cloudiness.

The tubes were further evaluated after 24 h and 48 h. In Batch I and II a slight decrease in the cloudiness could be detected but otherwise no major differences could be observed compared to the earlier readings.

Conclusion: All tablet formulations were dissolved in a similar manner in both the ISO-MRSA broth and in the Herogel medium. The reason for this is that both types of media were incubated in 35° simultaneously. The heat and the enzyme activity ensured that the gelatin became fluid rather quickly. The consistency of the medium is related to the dissolution rate.

Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims.

1. A kit for biological sampling comprising at least one collection vessel for receiving at least one sample, a growth medium and at least one enzyme, wherein the vessel has a closure for closing an open end of the vessel, the growth medium contained in the vessel is a gel based medium separated from the at least one enzyme and said at least one enzyme is an enzyme capable of digesting the gel based medium to a liquid growth medium upon coming into contact therewith.

2. The kit according to claim 1, comprising at least one selection agent.

3. The kit according to claim 2, wherein the at least one selection agent is an antibiotic.

4. The kit according to claim 1, wherein the vessel is adapted to comprise the at least one selection agent and/or the at least one enzyme and to release said at least one selection agent and/or the at least one enzyme substantially simultaneously with deposition of the sample in the collection vessel.

5. The kit according to claim 1, comprising a collection swab.

6. The kit according to claim 1, wherein the at least one selection agent is in powder form.

7. The kit according to claim 1, wherein the at least one enzyme is in powder form.

8. The kit according to claim 1, wherein the at least one selection agent is in liquid form.

9. The kit according to claim 1, wherein the at least one enzyme is in liquid form.

10. The kit according to claim 1, wherein the growth medium comprises gelatine.

11. The kit according to claim 10, wherein the concentration of gelatine is not more than about 50 mg/ml growth medium.

12. The kit according to claim 1, wherein the enzyme is a gelatine digesting enzyme.

13. The kit according to claim 1, wherein the at least one selection agent is suitable for selection of methicillin resistant Staphylococcus aureus (MRSA).

14. The kit according to claim 1, wherein the at least one selection agent is suitable for selection of extended spectrum β lactamase (ESBL) producing bacteria.

15. The kit according to claim 1, wherein the at least one selection agent is suitable for selection of Vancomycin resistant enterococci (VRE).

16. The kit according to claim 1, wherein the at least one selection agent is suitable for selection of Bordetella pertussis.

17. The kit according to claim 1, wherein the at least one selection agent is suitable for selection of Neisseria gonorreae.

18. The kit according to claim 1, wherein the at least one selection agent is suitable for selection of Myco-/Ureaplasma.

19. The kit according to claim 1, wherein the at least one selection agent is suitable for selection of Mycoplasma pneumoniae.

20. The kit according to claim 1, wherein the at least one selection agent is suitable for selection of a bacterial strain selected from the group consisting of enterohemorragic E. coli (EHEC), enterotoxigenic E. coli (ETEC), enteroinvasive E. coli (EIEC), enteropathogenic E. coli (EPEC) and enteroaggregative E. coli (EAggEC).

21. A method for selection and enrichment of microbes in a sample collected from a subject wherein selection and enrichment of microbes start in the collection vessel, wherein the method comprises the following steps:
collecting at least one microbial sample in a collection vessel comprising a growth medium, wherein the growth medium is a gel based growth medium,
adding at least one enzyme to the collection vessel, said enzyme dissolving said gel based medium to a liquid medium, and
selecting for said microbes in the collection vessel before analysis.

22. The method according to claim 21, wherein at least one selection agent is added to the collection vessel and said at least one selection agent is dissolved in the growth medium and is chosen for selection of a specific microbe.

23. The method according to claim 22, wherein the at least one selection agent and the at least one enzyme are added to the collection vessel substantially simultaneously with the sampling and deposition of the sample in the collection vessel.

24. The method according to claim 21, wherein the growth medium comprises gelatine.

25. The method according to claim 21, wherein the enzyme is a gelatine digesting enzyme.

26. The method according to claim 21, wherein the collection vessel is incubated at a temperature between about 35-40° C. during the selection.

27. The method according to claim 22, wherein the at least one selection agent is for selection of one or more of the following:
methicillin resistant Staphylococcus aureus (MRSA),
extended spectrum β lactamase (ESBL) producing bacteria,
Vancomycin resistant enterococci (VRE),
Bordetella pertussis,
Neisseria gonorreae,
Myco-/Ureaplasma, and
Mycoplasma pneumoniae.

28. A method for selection microbes in a sample, wherein a kit according to claim 1 is used.

29. A collection vessel for use in the method according to claim 21, said vessel having a closure for closing an open end of the vessel and said vessel has means for releasing the at least one compound, such as the at least one enzyme and/or the at least one selection agent, immediately after or simultaneously with the deposition of the biological sample in the collection vessel.

30. A collection vessel, comprising a closure for closing an open end of the vessel and wherein said vessel has means for releasing at least one compound immediately after or simultaneously with the deposition of the biological sample in the collection vessel.

31. The collection vessel according to claim 29, wherein said collection vessel has a carrier or support containing the at least one compound.

32. The collection vessel according to claim 29, wherein the closure is adapted to contain the at least one compound.

33. The collection vessel according to claim 29, wherein the collection vessel has elastic/springing means for holding a collection swab in a specific position after deposition of a sample in the collection vessel.

34. The collection vessel according to claim 33, wherein the elastic/springing means carries the at least one compound prior to deposition of a biological sample.
